(19) United States
(12) Plant Patent Application Publication
(10) Pub. No.: US 2017/0290228 P1
(43) Pub. Date: Oct. 5, 2017

(54) Dianthus plant named 'KLEDP15179'

(50) ;

(71) Applicant: Klemm+Sohn GmbH & Co. KG, Stuttgart (DE)

(72) Inventors: Nils Klemm, Stuttgart (DE); Lena Gutbrodt, Bempfingen (DE)

(73) Assignee: Klemm+Sohn GmbH & Co. KG, Stuttgart (DE)

(21) Appl. No.: 14/999,100

(22) Filed: Mar. 30, 2016

(51) Int. Cl. A01H 5/02

(52) U.S. Cl. CPC A01H 5/0233
USPC PLT/273

(57) ABSTRACT

A new dianthus plant particularly distinguished by having salmon colored flowers, good branching, and medium vigor, is disclosed.

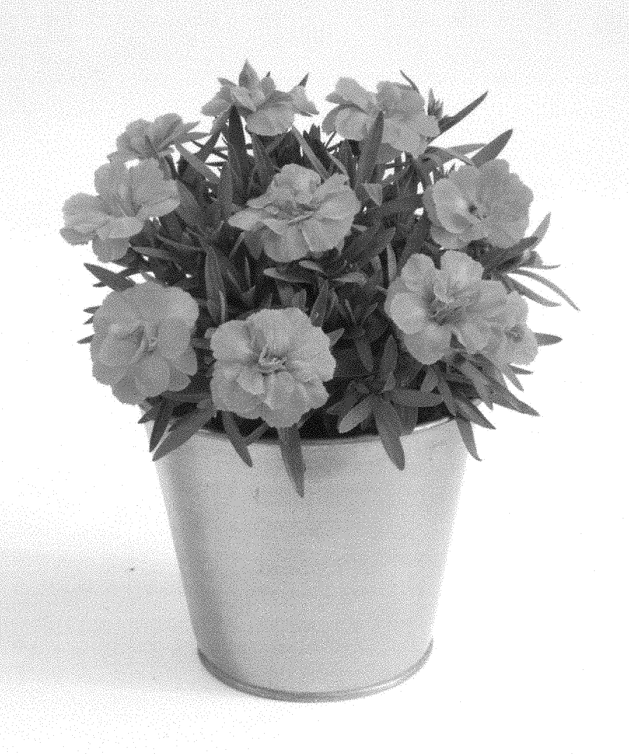

Genus and species: Dianthus caryophyllus.

Variety denomination: ‘KLEDP15179’.

### BACKGROUND OF THE NEW PLANT

The present invention comprises a new and distinct variety of dianthus, botanically known as Dianthus caryophyllus and hereinafter referred to by the variety name ‘KLEDP15179’. ‘KLEDP15179’ resulted from a whole plant mutation of ‘DCP-2010-1006’ (unpatented) identified in March 2010 in Stuttgart, Germany.

In July of 2010 ‘KLEDP15179’ was first vegetatively propagated by stem cuttings. ‘KLEDP15179’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.
1. Salmon colored flowers;
2. Good branching; and
3. Medium vigor.

### DESCRIPTION OF THE PHOTOGRAPH

This new Dianthus plant is illustrated by the accompanying photograph which shows an overall view of the plant. The colors shown are as true as can be reasonably obtained by conventional photographic procedures.

The photograph is of a plant that is 12 weeks-old grown in a greenhouse in Stuttgart, Germany in April 2014.

### DESCRIPTION OF THE NEW VARIETY

The following detailed descriptions set forth the distinctive characteristics of ‘KLEDP15179’. The data which define these characteristics were collected from asexual reproductions carried out in Stuttgart, Germany. Data was collected on 6 month-old plants grown in a glass greenhouse in Stuttgart, Germany in March 2016. Color references are to The R.H.S. Colour Chart of The Royal Horticultural Society of London (R.H.S.), 5^{th} edition (2007).
Classification:
   Family.—Caryophyllaceae.
   Species.—Dianthus caryophyllus.
   Common name.—Pot carnation.
   Variety.—‘KLEDP15179’.
Parentage: Mutation of ‘DCP-2010-1006’ (unpatented).
Propagation:
   Type.—Stem cuttings.
   Time to produce a rooted young plant.—14 to 16 days.
   Root description.—White, fine.
   Rooting habit.—Lanceolate.
Plant description:
   Plant form.—Potted plant, upright.
   Plant height.—13.0 cm to 15.0 cm.
   Plant diameter or spread.—16.0 cm to 20.0 cm.
Lateral branches:
   Length.—8.0 cm to 10.0 cm.
   Diameter.—0.3 cm to 0.5 cm.
   Internode length.—2.0 cm to 3.0 cm.
   Node length.—0.3 cm to 0.4 cm.
   Node diameter.—0.4 cm.
   Texture.—Smooth.
   Color.—RHS 137A.
   Branch.—Solid.
Foliage description:
   Arrangement.—Opposite.
   Length.—4.0 cm to 6.0 cm.
   Width.—0.6 cm to 0.8 cm.
   Shape.—Lanceolate.
   Apex.—Acute.
   Base.—Obtuse.
   Margin.—Entire.
   Texture (both surfaces).—Smooth.
   Venation pattern.—Palmate.
   Color.—Immature leaves: Upper surface: RHS 143A Lower surface: RHS 144A Mature leaves: Upper surfaces:RHS 139A Lower surface:RHS 137A Venation: RHS 139A.
Flower description:
   Flower type and habit.—Round, double, small sprays.
   Fragrance.—Present.
   Natural flowering season.—Spring to fall.
   Position of flowers relative to the foliage.—Slightly above.
   Number of flowers per spray.—3 to 4.
   Flower longevity.—10 to 14 days.
   Flower buds.—Length: 2.0 cm Diameter: 0.8 cm to 1.0 cm Shape: Elliptic Color: RHS 137C and RHS 144A Extrusion of the styles at bud stage: Absent.
   Corolla.—Quantity of petals/arrangement: Approximately 15, arranged in several whorls Corolla height: 1.5 cm to 2.0 cm Petal length: 3.0 cm Petal width: 1.5 cm to 2.0 cm Petal shape: Spoon shaped Petal apex: Obtuse Petal base: Acute Petal margin: Crenate Depth of petal margin: Medium Petal texture (both surfaces): Smooth Petal color immature: Upper surface: RHS 52B Lower surface: RHS 52D Petal color mature: Upper surface: RHS 51B Lower surface: RHS 51D Calyx: Quantity of sepals/arrangement: 5, fused Shape: Campanulate Calyx height: 1.8 cm to 2.0 cm Calyx diameter, at the apex: 0.8 cm to 1.0 cm Sepal texture (both surfaces): Smooth Sepal color: Upper surface: RHS 137A Lower surface: RHS 137D.
Peduncles:
   Length.—10.0 cm to 14.0 cm.
   Diameter.—0.2 cm.
   Strength.—Moderate.
   Texture.—Smooth.
   Color.—RHS 137A.
Reproductive organs:
   Stamens.—Filament length: 0.4 cm to 0.6 cm Filament color: RHS 155B Anther length: 0.1 cm Anther diameter: 0.05 cm Anther color: RHS 19B Pollen amount: Abundant.
   Pistils.—Quantity: 1 Pistil length: 0.1 cm Style length: 1.6 cm to 1.8 cm Style color: RHS 56D Stigma shape: Filamentous Stigma color: RHS 55A Ovary size: 0.4 cm to 0.6 cm Ovary shape: Oval Ovary color: RHS 144B and RHS 145B
Fruit and seed: Seed sometimes observed.
Disease and insect/pest resistance: No special observations were made.
Comparison with parental line and known variety: ‘KLEDP15179’ is a distinct variety of dianthus. ‘KLEDP15179’ is distinguished from its parent as described in Table 1:

TABLE 1
Comparison with Parental Line

| | Variety | Parent |
|---|---|---|
| Trait | ‘KLEDP15179’ | ‘DCP-2010-1006’ |
| Flower color | Salmon | Red |

‘KLEDP15179’ is most similar to the commercial dianthus plant named ‘Oscar’ also known as ‘KLEDP07089’ (unpatented). Differences between the two varieties are described in Table 2:

TABLE 2
Comparison with Similar Variety

| | Variety | Commercial line |
|---|---|---|
| Trait | ‘KLEDP15179’ | ‘Oscar’ |
| Flower color | Salmon | Pink with Purple eye |

We claim:

1. A new and distinct variety of dianthus plant named ‘KLEDP15179’ as illustrated and described herein.
